(19) United States
(12) Patent Application Publication
(10) Pub. No.: US 2013/0252337 A1
(43) Pub. Date: Sep. 26, 2013

(54) SUBSTRATES WITH MICROMETER AND NANOMETER SCALE STIFFNESS PATTERNS FOR USE IN CELL AND TISSUE CULTURING AND A METHOD FOR MAKING SAME

(71) Applicant: Shengyuan Yang, West Melbourne, FL (US)

(72) Inventor: Shengyuan Yang, West Melbourne, FL (US)

(21) Appl. No.: 13/426,593

(22) Filed: Mar. 21, 2012

(51) Int. Cl. C12N 5/071; B23P 17/04

(52) U.S. Cl. 435/395; 29/428

(57) ABSTRACT

A substrate comprising a base, first regions having a first stiffness value and disposed above the base, second regions having a second stiffness value disposed above the base, wherein the first stiffness value is different from the second stiffness value, and wherein a portion of one or more of the first regions and a portion of one or more of the second regions is exposed on an upper surface of the substrate.

### FIELD OF THE INVENTION

The present invention is directed generally to substrate material on which cells and tissues are cultured and more particularly to such substrates with various localized stiffness and geometric patterns at micrometer and nanometer scales.

### BACKGROUND OF THE INVENTION

Cells survive in a mechanical environment of a tissue to which the cells belong. It has been shown that cell behaviors are extremely sensitive to the stiffness of the substrate(s) on which the cells grow. For example, cell movement can be guided solely by the rigidity or stiffness of the substrates: epithelial cell traction forces are proportional to substrate rigidity; matrix elasticity directs stem cell lineage specification; on microstructured anisotropic substrates, epithelial cells migrate along the direction of greatest stiffness; within a range of stiffness values spanning that of soft tissues, fibroblasts tune their internal stiffness to match that of their substrate.

However, the underlying mechanism through which substrate stiffness (SS) directs these cell behaviors remains largely unknown. The present invention discloses substrates that can be used to study how the cells sense the SS locally and how the cells integrate the local SS information to reach a decision that controls global cell behavior.

### BRIEF DESCRIPTION OF THE DRAWINGS

The foregoing and other features of this invention will be apparent from the following more particular description of the invention, as illustrated in the accompanying drawings, in which like reference characters refer to the same parts throughout the different figures.

FIG. 1 is a front view of a substrate according to one embodiment of the invention.

FIG. 2 is an orthographic view of a frame for use in forming a substrate according to a second embodiment of the invention.

FIGS. 3, 4, 5 and 6 are front views of substrates according to additional embodiments of the invention.

### DETAILED DESCRIPTION OF THE INVENTION

Before describing in detail the particular substrates for use in various applications, including cell and tissue culturing, with varying stiffness and geometric patterns on an exposed surface thereof, in accordance with the present invention and methods for making same, it should be observed that the present invention resides primarily in a novel combination of elements and method steps. Accordingly, these elements have been represented by conventional elements in the drawings, showing only those specific details that are pertinent to the present invention so as not to obscure the disclosure with structural details that will be readily apparent to those skilled in the art having the benefit of the description herein.

The following embodiments are not intended to define limits as to the structure or methods of the invention but only to provide exemplary constructions. The described embodiments are permissive rather than mandatory and illustrative rather than exhaustive.

To better understand how cells and tissues interact with the substrate on which they are cultured and the mechanisms that control this interaction, it is desired to construct substrates with non-uniform localized stiffness patterns (including regions with at least two different stiffness values) and localized geometries on an exposed surface thereof. The stiffness patterns and geometries are at the micro- and nano-meter scale.

Generally, in the context of the present invention, micrometer and nanometer scale references include sub-millimeter dimensions and refer to dimensions in a range of less than about 1 nanometer to about 1 millimeter.

The embodiments of the present invention teach one or more substrates with stiffness patterns and geometric shapes at micrometer and nanometer scales and one or more processes for producing substrates with stiffness patterns and geometric shapes at micrometer and nanometer scales. These substrates are advantageous for use in growing and studying cell and tissue cultures. These processes and substrates have allowed the inventor to realize micromechanical control of cell and tissue development, which has crucial applications in tissue engineering and regenerative medicine.

The various stiffness patterns disclosed herein refer to regions of differing stiffness (regions of relative high, low and intermediate stiffness, for example) at an exposed surface of a substrate. As known by those skilled in the art, stiffness is the ratio of a force applied at a position on a surface of a material to a displacement of that position in the same direction as the applied force. The unit of surface stiffness is N/m (i.e., Newton/meter). The stiffness at each location on and within the substrate and the stiffness pattern depend on the Young modulus of the material(s) that comprise the substrate and the materials formed within and on a surface of the substrate, i.e., the pattern of those materials. In general, Young's modulus (or elastic modulus) is not the same as stiffness. The elastic modulus is a property of a material; stiffness is a property of a structure. Thus the stiffness depends on the structure material (i.e., Young's modulus of the material) and the shape of the structure.

As applied to the present invention, the high-stiffness structures generally comprise rigid materials (high Young's modulus) and the low-stiffness structures generally comprise soft or flexible materials (low Young's modulus).

The localized stiffness values of a structure in a given high and low stiffness pattern, can be routinely and accurately determined by finite element methods (FEM) and measured by atomic force microscopy (AFM), or other similar techniques known by those skilled in the art. Stiffness estimates, based on the Young modulus of the material and a general description of the surface geometric pattern, can be obtained by using simple, readily available mechanics formulae. These estimates are sufficiently accurate to allow a user to select the materials and stiffness patterns for any intended purpose.

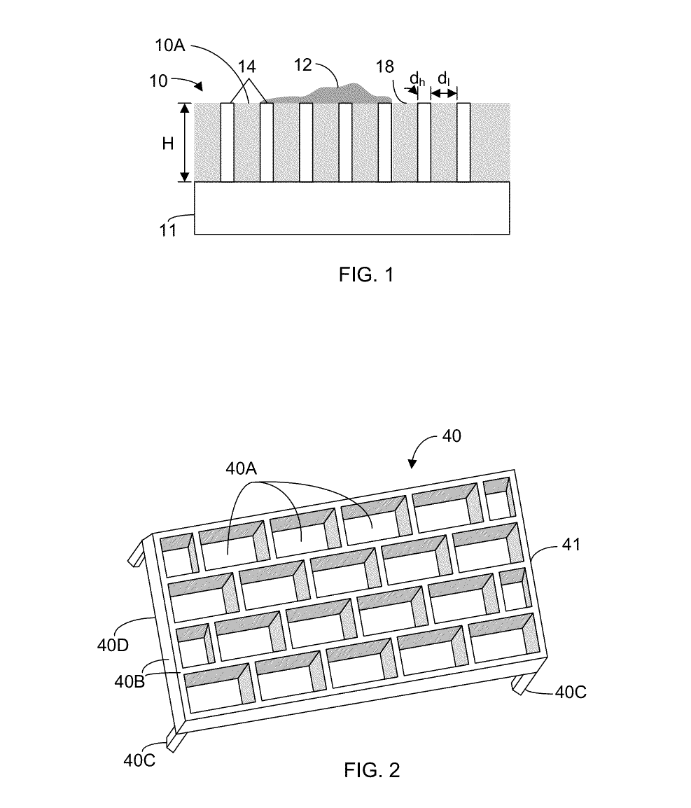

FIG. 1 illustrates a substrate 10 with a stiffness pattern according to one embodiment of the invention. The substrate 10 is formed overlying a plate, base or body 11, which can be constructed of any material (e.g., steel) that is compatible with the structures and materials to be formed above the body 11. The stiffness of the region 11 is not pertinent to the present invention. A cell or tissue portion 12 sets on an upper surface 10A of the substrate 10. In this embodiment, the substrate 10 comprises high stiffness regions 14 and low stiffness regions 18, both of which appear on the exposed surface 10A.

In other embodiments, regions with stiffness values other than the high and low stiffness values referred to, can be present within the substrate and/or on the exposed surface 10A. Also, regions having more than two stiffness values can be present within the substrate and/or on the exposed surface 10A. For example, certain regions can have a first stiffness value, other regions a second stiffness value, yet other regions a third stiffness value and still other regions a fourth stiffness value. Thus regions of many different stiffness values (i.e., at least two) can be formed in the substrate as the research objectives dictate. The present invention provides substrates exhibiting different stiffness values so that one can study the extent to which cells and tissues can differentiate between these different stiffness values in their growth processes.

A material of the high stiffness regions 14 may comprise glass formed as pillars or posts as illustrated. These pillars or posts extend approximately vertically (i.e., perpendicularly) from an upper surface of the region 11.

Generally, it is not necessary to quantify stiffness values for the high and low stiffness regions to effectively use the present invention. Instead, it is only necessary for the high and low stiffness regions to exhibit different stiffness values. Cells and tissues can differentiate between stiffness values and this is the only requirement for the present invention to function as described.

The high stiffness regions 14 are spaced apart from each other and according to this embodiment have approximately the same shape and size, although these features are not required according to the present invention. For example, the high stiffness regions may not be spaced at equidistant intervals and may not all have the same height H (see FIG. 1).

Young's moduli for the high and low stiffness regions are designated Eₕ and Eₗ, respectively. In an embodiment where the high stiffness regions are circular in cross-section, and thus in the shape of cylinders, a diameter of each high stiffness region is dₕ, and a distance between adjacent high stiffness regions is dₗ. The thickness of the substrate 10 is designated H.

The parameters Eₕ, Eₗ, dₕ and dₗ can be varied to produce substrates with different stiffness patterns at different scale sizes as required to meet the needs of the associated cell or tissue experiments.

The cell or tissue portion 12 cultured on the exposed surface 10A covers both the high and low stiffness regions 14 and 18. Since there are no empty spaces between the high and low stiffness regions 14 and 18 (at least in the embodiment of FIG. 1), this configuration allows one to study how the cell senses local variation of SS or differentiates the difference in stiffness between adjacent substrate regions. Using a substrate formed according to the teachings of the present invention, a researcher can control and detect the behaviors and the status of the cells and tissues, e.g., cell adhesion, spreading, migration, division, differentiation, cell-cell interactions, cell-cell communications, and the health status of the cells and tissues. Of course, it is expected that the user will select a substrate comprising an array of high and low stiffness regions to suit his/her research needs.

For example, by changing 4 and dₗ for fixed Eₕ and Eₗ stiffness values, the effect of the various stiffness values and their distribution permits study of cell sensitivities to local SS. Alternatively, by changing Eₕ and Eₗ for a fixed dₕ and dₗ values, the ability of a cell to differentiate local variation in SS can be studied. Thus quantitative control of cell behaviors can be realized by adjusting the values of Eₕ, Eₗ, dₕ, and dₗ, which is referred to as micro- and nano-patterning of the SS.

It is a challenge to physically realize the stiffness pattern in the substrate embodiment of FIG. 1 since the substrate must be thick enough (i.e., H much be large) so the low stiffness regions 18 (with a thickness of dₗ), can exhibit the desired low stiffness. Also, the low stiffness regions 18 can deform in response to a sufficient force and the high stiffness regions can also deform or deflect responsive to a sufficient force.

For example, if a polyacrylamide gel is used as a material for the low stiffness regions 18, according to the inventor's experience, gels with a thickness in a range of about 50-70 μm can typically achieve the variation in SS (from several hundred Pa to tens of kPa) that is required for accurate cell studies. Therefore, the desired value of H in FIG. 1 should be on the order of about 50-70 μm for the substrate 10 to have the desired effective stiffness patterns. Since the thickness of the substrate 10 also represents a height of the pillar-like high stiffness regions 14 in FIG. 1, it is further desired to fabricate pillars with heights on the scale of about 50-70 μm. But fabricating such pillars may be difficult, especially when the diameter of the pillars is reduced to micrometer and nanometer scales.

According to one fabrication technique, the high stiffness regions 14 (in one embodiment formed as pillars or posts) are formed from the base 11 using various semiconductor processing techniques. A soft polymer gel (the low stiffness regions 18) is poured or injected onto the pillar array or the pillar array is inserted into the soft polymer gel to form the illustrated substrate 10. The width dₗ and the depth (the out-of-plane dimension in FIG. 1) of the high stiffness pillars 18 (i.e., the cross-sectional dimensions), the pillar height and the distance between any adjacent pillars can be selected as desired and all are on the order of micrometer and nanometer scales.

According to a different embodiment, additional gel material can be added above an entire substrate (including a substrate formed from the suspended frame as described below) to fabricate a substrate with a variable gel thickness in different regions, allowing many different stiffness patterns to be realized.

Finite element models can be used to accurately predict and atomic force microscopy (AFM) and nano-indentations can be used to characterize the stiffness patterns of the substrate.

In another embodiment, instead of fabricating the pillars of FIG. 1, a suspended high stiffness frame (comprising a high stiffness material that will form the high stiffness regions when the process is completed) with a height of about 5-20 μm is first formed. Fabrication of such a frame can be routinely accomplished according to currently known processes. Openings or empty regions within the frame and beneath the frame when it is suspended from a surface by frame legs are filled with one or more low stiffness materials, such as soft polymer gels. The elements of the suspended frame (e.g., sidewalls defining the openings and the boundary of the frame) define the stiffness pattern in the final formed substrate. The height at which the frame is suspended and the thickness of the frame determine the gel thickness for the low stiffness regions.

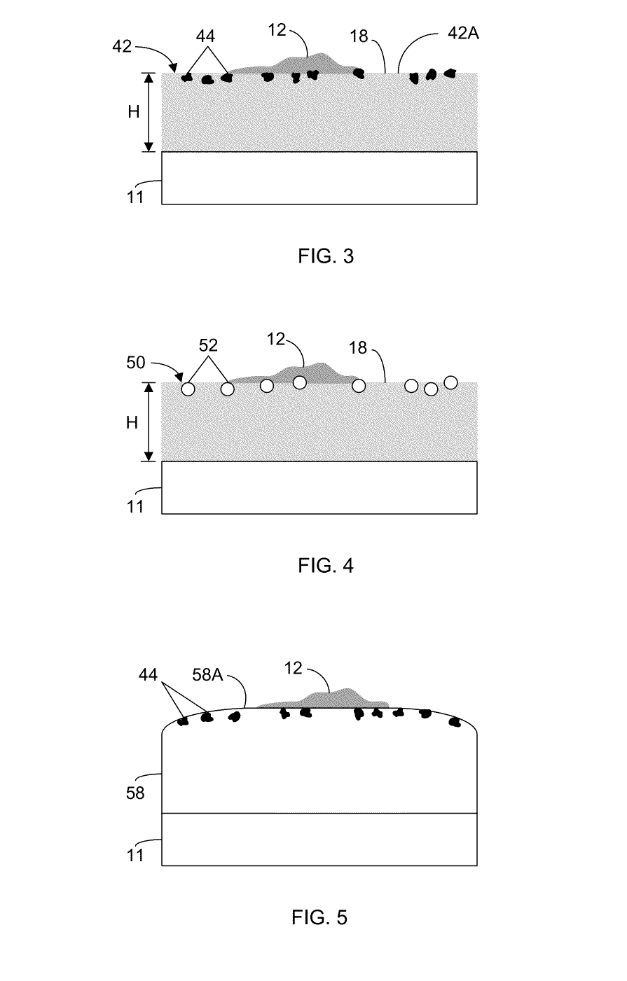

FIG. 2 illustrates a frame 40 fabricated, in one embodiment, from single-crystal silicon using a modified single-crystal reactive etching and metallization (SCREAM) process, which is known in the art. Various semiconductor and MEMS processes can be used to form the frame 40.

The frame 40 can also be fabricated by a process described in a paper entitled “Micromachined Force Sensors for the Study of Cell Mechanics”, by Shengyuan Yang and Taher Saif, Review of Scientific Instruments 76, 044301 (2005); doi: 10.1063/1.1863792, which is also incorporated by reference.

The frame 40 comprises a border 41 bounding a plurality of rectangular openings 40A separated by upstanding wall surfaces 40B. Other frames of different sizes, geometries, and shapes, compartment patterns, compartment wall thicknesses, and compartment shapes (i.e., any closed polygonal shape), and wall patterns can be used according to the present invention. These frame parameters can be selected according to the requirements of the cell and tissue growth experiments. For example, although the frame openings 40A are illustrated as uniformly distributed and rectangular in shape in FIG. 2, such is not required and may not be desired for studying certain cell and tissue cultures. The frame 40 is supported by legs 40C.

In one embodiment, the thickness of the silicon structures of the frame 40 can be reduced by forming silicon dioxide (SiO₂) by thermal oxidation of the silicon, followed by hydrofluoride (HF) etching of the SiO₂. Using this technique (or another semiconductor processing or MEMS processing techniques) continuous frame structures with desired stiffness patterns at micrometer and nanometer scales can be obtained.

The frame structures illustrated in FIG. 2 can be formed from a relatively high-stiffness material (such as silicon) and the openings 40A filled with a relatively low stiffness material. This process can be performed by injecting or dispensing the low stiffness material into the openings 40A or by immersing the frame 40 in the low stiffness material such that the low stiffness material fills the openings 40A. In one embodiment the low stiffness material within the openings 40A comprises a polyacrylamide gel. Compared to these gel regions, the high stiffness regions of the frame 40 may be considered infinitely stiff.

In one embodiment a material fills a volume between a lower surface 40D of the frame 40 and a surface (not shown in FIG. 2) on which the frame 40 rests. For example, in one embodiment this material comprises the same material that fills the openings 40A.

Polydimethylsiloxane (PDMS) (a material having a high value of Young's modulus) can also be used to fabricate the frame 40. Since the Young's modulus of PDMS can be varied from tens of kPa to 2.5 MPa or higher and PDMS structures down to 1-2 μm can be fabricated, PDMS frames may be preferable to silicon frames for use in certain applications. The modulus can be varied by changing the concentration of the various constituent elements or by changing the constituent elements of the PDMS.

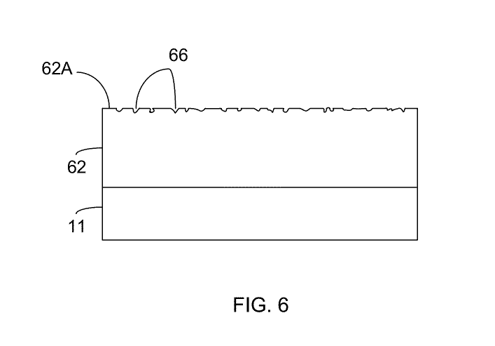

FIG. 3 illustrates a substrate 42 formed using micro- and nano-composite concepts such that components of the substrate 42 produce a desired stiffness pattern. In this embodiment the rigid or high stiffness materials are in the form of particles 44. The particles 44, which are at micrometer and nanometer scales, are dropped (randomly) onto and then embedded in the soft (i.e., low stiffness) materials to form stiffness patterns. The particles may be randomly shaped and therefore all particles may not be the same shape and size. The use of randomly shaped particles also permits study of cell and tissue responses to variously localized-shaped curves on an exposed surface 42A of the substrate 42.

FIG. 4 illustrates a substrate 50 formed with various stiffness patterns using micrometer and nanometer composites and processes. In this embodiment the rigid (high stiffness) materials are in the form of spheres or cylinders 52. The spheres or cylinders (at micrometer and nanometer scales) are dropped onto and then embedded in the soft low stiffness materials to form a stiffness pattern.

FIG. 5 illustrates another embodiment in which a low stiffness region 58 presents a curved exposed surface 58A, unlike the relatively planar exposed surfaces of FIGS. 1, 3 and 4. Although FIG. 5 illustrates the particles 44, other embodiments, not illustrated, comprise the pillars or posts of high the high stiffness regions 14 of FIG. 1 or the spheres or cylinders 52 of FIG. 4.

FIG. 6 illustrates another embodiment comprising nano-indentations 66 in an exposed surface 62A of a material layer 62, wherein the material layer 62 has any stiffness value. The nano-indentations 66 generally have a depth and an opening on the order of nanometers, i.e., less than 1 nanometer to about 1 millimeter. The FIG. 6 embodiment may further comprise the pillars or posts of high the high stiffness regions 14 of FIG. 1, the particles 44 of FIG. 3, or the spheres or cylinders 52 of FIG. 4.

Thus the described embodiments depict substrates with different and localized exposed surface shapes and geometries and localized stiffness properties within the substrate, especially different stiffness properties and geometries presented on an exposed surface of the substrate.

The present invention allows the fabrication of widely varying surface stiffness patterns. In the frame embodiment of FIG. 2, surface planarity is also assured by the planarity of the suspended frame and the excellent rheological properties of the gels.

In another embodiment, the high-stiffness particles can be placed within the low stiffness material according to a desired pattern of high stiffness regions, instead of randomly distributed as described above.

The following paragraphs describe methods and techniques for preparing substrates and the high and low stiffness regions according to the various presented embodiments.

The polyacrylamide-micropillar composite substrate as illustrated in FIG. 1 can be prepared according to either of the following two methods.

Method 1:

1) Place a removable sterilized cover glass on the inside bottom surface of a Petri dish.

2) Place a micropillar structure (i.e., a structure in which micropillars, (e.g., the high stiffness regions 14 of FIG. 1) have been formed) on top of the cover glass.

3) Mix 40 g of acrylamide powder to 100 ml of de-ionized water to create 40% w/v (weight per volume) solution.

4) Mix 2 g of bisacrylamide powder to 100 ml of de-ionized water to create 2% w/v solution.

5) Pipette 1000 ul of acrylamide solution, 200 ul of bisacrylamide solution, 50 ul of 1M HEPES (4-(2-hydroxyethyl)-1-piperazineethanesulfonic acid) solution and 3750 ul of de-ionized water into a beaker and mix thoroughly.

6) Degas the solution for 20 minutes to remove oxygen, which inhibits polymerization.

7) Add 1 g of ammonium persulfate to 10 ml of de-ionized water to create 10% w/v solution.

8) Pipette 20 ul of ammonium persulfate solution and 20 ul of TEMED (Tetramethylethylenediamine) into beaker and swirl gently.

9) Immediately pipette 25 ul of the final solution uniformly onto the micropillar structure and place a cover glass sheet on top of the micropillar structure within the Petri dish. Apply a downward pressure to the glass so that it rests atop the micropillar structure.

10) Allow the solution to polymerize for about 30 minutes.

11) Rinse the substrate with PBS (Phosphate Buffered Saline) and gently remove the cover glass.

Method 2:

1) Place a sterilized cover glass on a bottom inside surface of a Petri dish.

2) Mix 40 g of acrylamide powder to 100 ml of de-ionized water to create a 40% w/v solution.

3) Mix 2 g of bisacrylamide powder to 100 ml of de-ionized water to create 2% w/v solution.

4) Pipette 1000 ul of acrylamide solution, 200 ul of bisacrylamide solution, 50 ul of 1M HEPES (4-(2-hydroxyethyl)-1-piperazineethanesulfonic acid) solution and 3750 ul of de-ionized water into a beaker and mix thoroughly.

5) Degas the solution for 20 minutes to remove oxygen, which inhibits polymerization.

6) Add 1 g of ammonium persulfate to 10 ml of de-ionized water to create a 10% w/v solution.

7) Pipette 20 ul of ammonium persulfate solution and 20 ul of TEMED (Tetramethylethylenediamine) into beaker and swirl gently.

8) Immediately pipette 25 ul of the final solution onto the cover glass evenly and place the micropillar structure on top with the micropillars facing down. Apply downward pressure to the micropillar structure to permit it to rest atop the cover glass and not float on the solution.

9) Allow the solution to polymerize for 30 minutes.

10) Rinse the substrate with PBS (Phosphate Buffered Saline) and gently remove the cover glass.

The polyacrylamide-silicon composite substrate (the embodiments associated with FIG. 2) can be prepared according to either of the following two methods.

Method 1:

1) Place sterilized cover glass on bottom of Petri dish.

2) Place silicon frame on top of cover glass.

3) Mix 40 g of acrylamide powder to 100 ml of de-ionized water to create 40% w/v solution.

4) Mix 2 g of bisacrylamide powder to 100 ml of de-ionized water to create 2% w/v solution.

5) Pipette 1000 ul of acrylamide solution, 200 ul of bisacrylamide solution, 50 ul of 1M HEPES (4-(2-hydroxyethyl)-1-piperazineethanesulfonic acid) solution and 3750 ul of de-ionized water into a beaker and mix thoroughly.

6) Degas the solution for 20 minutes to remove oxygen which inhibits polymerization.

7) Add 1 g of ammonium persulfate to 10 ml of de-ionized water to create 10% w/v solution.

8) Pipette 20 ul of ammonium persulfate solution and 20 ul of TEMED (Tetramethylethylenediamine) into beaker and swirl gently.

9) Immediately pipette 25 ul of the final solution onto silicone substrate (i.e., the frame 40 of FIG. 2) evenly and place a cover glass on top. Apply a downward pressure to the over glass to ensure that the cover glass rests on top of the silicon substrate.

10) Let solution polymerize for 30 minutes.

11) Rinse substrate with PBS (Phosphate Buffered Saline) and gently remove the cover glass.

Method 2:

1) Put sterilized cover glass on bottom of Petri dish.

2) Mix 40 g of acrylamide powder to 100 ml of de-ionized water to create 40% w/v solution.

3) Mix 2 g of bisacrylamide powder to 100 ml of de-ionized water to create 2% w/v solution.

4) Pipette 1000 ul of acrylamide solution, 200 ul of bisacrylamide solution, 50 ul of 1M HEPES (4-(2-hydroxyethyl)-1-piperazineethanesulfonic acid) solution and 3750 ul of de-ionized water into a beaker and mix thoroughly.

5) Degas the solution for 20 minutes to remove oxygen which inhibits polymerization.

6) Add 1 g of ammonium persulfate to 10 ml of de-ionized water to create 10% w/v solution.

7) Pipette 20 ul of ammonium persulfate solution and 20 ul of TEMED(Tetramethylethylenediamine) into beaker and swirl gently.

8) Immediately pipette 25 ul of the final solution onto the cover glass evenly and place the frame 40 on the final solution. Apply downward pressure to the frame using another cover glass so that the frame rests on top of the cover glass and does not float on the solution.

9) Let the solution polymerize for about 30 minutes.

10) Rinse substrate with PBS (Phosphate Buffered Saline) and remove cover glass gently.

As can now be appreciated, the structure of the present invention can be used to advantageously mechanically manipulate the biological direction in which a cell grows. Control of that growth direction and process allows careful study of the cell and tissue growth process, both normal cell growth and abnormal cell growth.

Although the various embodiments of the invention have been described for application to cell and tissue cultures, the teachings of the inventions can be employed in any situation or application where a surface, interface, micro-composite structure or nano-composite structure that presents different localized stiffness values and different localized geometries on an external surface of the structure is desired.

Although the invention has been shown and described with respect to certain preferred embodiments, it is obvious that equivalent alterations and modifications will occur to those skilled in the art upon reading and understanding this specification and the annexed figures. In particular regard to the various elements and functions described above, the terms used to describe such elements and functions are intended to correspond, unless otherwise indicated, to any element or function that performs the specified function (i.e., that is functionally equivalent), even though not structurally equivalent to the disclosed structure that performs the function in the illustrated exemplary embodiments of the invention. In addition, while a particular feature of the invention may have been disclosed with respect to only one embodiment, such feature may be combined with one or more other features of the other embodiments as may be desired and advantageous for any given or particular application.

1. A substrate comprising:
a base;
first regions having a first stiffness value and disposed above the base;
second regions having a second stiffness value disposed above the base, wherein the first stiffness value is different from the second stiffness value; and
wherein a portion of one or more of the first regions and a portion of one or more of the second regions is exposed on an upper surface of the substrate.

2. The substrate of claim 1 for use in cell and tissue studies and culturing, wherein the upper surface serves as an interface between the first and second regions and a cell.

3. The substrate of claim 1 wherein the first regions and the second regions are configured with second regions disposed between consecutive spaced-apart first regions.

4. The substrate of claim 1 further comprising third regions having a third stiffness value, wherein the third stiffness value is different from both the first and second stiffness values.

5. The substrate of claim 1 wherein the first stiffness value is greater than the second stiffness value and each one of the first regions comprises one or more of a pillar extending approximately perpendicularly from an upper surface of the base, an irregularly-shaped particle, a sphere, and a cylinder having an axis oriented other than approximately perpendicularly to an upper surface of the base.

6. The substrate of claim 5 wherein the pillar has a circular or a rectangular cross-section.

7. The substrate of claim 1 wherein each one of the first regions has at least one dimension in a range of about range of about 1 nanometer to about 1 millimeter and each one of the second regions has at least one dimension in a range of about 1 nanometer to about 1 millimeter.

8. The substrate of claim 1 wherein the first stiffness value is greater than the second stiffness value, and wherein a material of the second regions comprises one of polyacrylamide gel and a polymer, and wherein a material of the first regions comprises one of silicon, glass and polydimethylsiloxane.

9. The substrate of claim 1 wherein the second regions comprise a material layer in contact with the base and the first regions comprise a plurality of detached structures disposed within the material layer and having a surface thereof exposed on an exposed surface of the material layer.

10. The substrate of claim 1 wherein the first regions comprise elements of a frame having a border and a plurality of openings within the border and upstanding walls defining the openings, wherein at least one of the border and the upstanding sidewalls comprise the first regions, the second regions within the openings, and wherein the first stiffness value is greater than the second stiffness value.

11. The substrate of claim 10 wherein each one of the plurality of openings has a polygonal shape.

12. The substrate of claim 10 wherein the plurality of openings have different areas in a cross-sectional plane.

13. The substrate of claim 1 wherein an upper surface of the substrate comprises a planar surface or a curved surface.

14. The substrate of claim 1 wherein an upper surface of the substrate defines nano-indentations.

15. A method for forming a substrate comprising:
presenting a base;
forming high stiffness regions above an upper surface of the base;
forming low stiffness regions between the high stiffness regions; and
wherein a portion of one or more of the low stiffness regions and a portion of one or more of the high stiffness regions is exposed on an upper surface of the substrate.

16. The method of claim 15 wherein the step of forming low stiffness regions comprises dispensing material having a low stiffness between the high stiffness regions.

17. The method of claim 15 wherein the high stiffness regions extend from an upper surface of the base and the step of forming the low stiffness regions comprises immersing the base and the high stiffness regions in a low stiffness material and removing the base and the high stiffness regions from the low stiffness material, wherein after the step of removing, the low stiffness material is present between the high stiffness regions.

18. A method for forming a substrate comprising:
forming a frame having openings therein and upstanding side walls defining the openings, the frame comprising a high stiffness material; and
forming low stiffness regions within the openings.

19. The method of claim 18 wherein the step of forming the low stiffness regions comprises dispensing material having a low stiffness within the openings.

20. The method of claim 18 wherein the step of forming the low stiffness regions comprises immersing the frame in a low stiffness material and removing the frame from the low stiffness material.
